(19) United States
(12) Patent Application Publication
(10) Pub. No.: US 2004/0141874 A1
(43) Pub. Date: Jul. 22, 2004

(54) System and apparatus for ozonating air and water for animal confinement houses

(76) Inventor: Phillip Mullinax, Dahlonega, GA (US)

Correspondence Address: TECHNOPROP COLTON, L.L.C., P O BOX 567685, ATLANTA, GA, 311567685

(21) Appl. No.: 10/342,975

(22) Filed: Jan. 15, 2003

(51) Int. Cl. A61L 9/015; C02F 1/78

(52) U.S. Cl. 422/4; 422/5; 422/28; 422/123; 119/447; 119/420; 119/72; 119/677; 210/760

(57) ABSTRACT

A combination system for ozonating the air and water provided to an animal confinement house, such as a poultry house, to provide breathing air and drinking water with less viruses, bacteria and contaminants to the growing animals, such as poultry, using an air ozonation system to provide ozonated air to the animal confinement house and a water ozonation system to provide ozonated water to the animal confinement house, preferably utilizing a single ozone reactor to provide ozone to both the air ozonation system and the water ozonation system.

### BACKGROUND OF THE INVENTION

1. Technical Field

The present invention relates generally to the field of raising healthier animals in animal confinement houses and relates more specifically to the field of raising healthier poultry by balancing the poultry house environment and reducing the amount of viruses, bacteria and other contaminants in the poultry house air and drinking water by treating the air and water entering the poultry house with ozone.

2. Prior Art

As the consumption of poultry products in the US and throughout the world increases, it is imperative to raise healthier birds, both from the public health standpoint and from the corporate profit standpoint. Healthier birds will result in fewer people becoming ill from eating poultry, thus increasing the public health, and will result in fewer pounds of birds going to waste, thus increasing the profitability of poultry raising companies. Many different systems, methods and devices have been employed to increase the yield of birds.

A poultry house often has high concentrations of noxious gases resulting from the natural life processes of the poultry. For example, respiration and decomposition of excess food and waste products can produce such noxious gases and other contaminants. Further, the excess food and waste products can serve as a breeding ground for viruses and bacteria and can adversely affect the physiology of the birds resulting in slower growth and lower yield.

The use of exhaust fans to reduce the concentrations of noxious gases in poultry houses is well known. However, exhaust fans can increase the cost of heating and cooling the building. The use of disinfectant chemicals to destroy viruses and bacteria in poultry houses also is well known. However, the use of such chemicals have a number of drawbacks, including high cost and the dangerous possibility of such chemicals finding their way into the food chain, contaminating the birds, and being consumed by humans.

Commercial poultry producers are interested in low feed conversion ratios and low bird mortality rates. Feed conversion ratio is the amount of feed consumed relative to the amount of bird produced, and bird mortality rate is the number of birds dying prior to harvesting relative to the number of harvested birds. Commercial poultry producers therefore attempt to minimize feed conversion ratios and minimize bird mortality rate, each of which can lower production costs, increase yield rates, and create greater profits. Thus, healthier birds can help achieve this goal.

A factor affecting both feed conversion ratios and bird mortality rates is the quality of the environment in the poultry house, including the air supply and the water supply provided to the poultry house, and the air and water provided to the poultry. Obviously, a higher quality environment will result in a higher bird quality, both through higher feed conversion ratios and lower mortality rates, resulting in a more cost efficient operation and higher profits. One method of increasing air quality within the poultry house is through the ozonation of the ambient air provided to the poultry house. Similarly, one method of increasing water quality within the poultry house is through the ozonation of the ambient water provided to the poultry house.

While air and water treatment systems that use ozonation as a disinfecting agent have been used previously, these systems to date have been separate systems used for specific treatment purposes. For example, U.S. Pat. No. 6,106,731 to Hayes discloses a system and method for ozonating water for animal confinement houses comprising taking pressurized water from a water main, maintaining the water under a pressure less than the original water pressure but greater than atmospheric pressure, ozonating the water under pressure, and, ultimately, providing the ozonated water to the animal confinement house for consumption. Although the Hayes '731 system is step- and equipment-intensive, it does serve as a means for providing ozonated water to, for example, a poultry house. Much simpler water ozonating systems are available and can be utilized in the present invention's system.

U.S. Pat. No. 6,156,268 to Curry discloses an ozone distribution system for an enclosed space, in particular, an air ozone system for introducing ozone into an animal confinement house. Specifically, the Curry '268 system comprises a double tube system in which ozonated air is mixed with ambient air proximal to a duct register within the animal confinement house. Although the Curry '268 system also is step- and equipment-intensive, it does serve as a means for providing ozonated air to, for example, a poultry house. Much simpler air ozonating systems are available and can be utilized in the present invention's system.

U.S. Pat. No. 6,325,971 to Hayes discloses a method and system for disbursing ozone into a poultry house comprising means for directing ozone under and through the poultry house litter. The Hayes '971 patent is directed specifically to a system for reducing pathogens in a poultry house by disinfecting the poultry house litter.

Thus, from both a basic health standpoint and a commercial profit standpoint, it can be seen that providing a clean living environment for animals while they are being raised can result in a cleaner and healthier animal. As discussed previously, cleaner and healthier animals result in cleaner and healthier food products, as well as more or a greater yield of food products, which is to everyone's advantage. However, as can be seen, a complete and combined system for ozonating arguably the two most important necessities for raising animals—air and water—is not disclosed. Therefore, there is a need for an ozone air and water treatment system that is relatively simple in design and economical to operate. It is to this need and other needs that the present invention is directed.

### BRIEF SUMMARY OF THE INVENTION

The present invention harnesses nature's ozone with a method and system for introducing the ozone to an animal confinement house, such as a poultry house, to help rebalance environments within the animal confinement house. The present invention improves animal wellness, reduces environmental impact and offensive odors, improves feed conversion and animal weight, eliminates litter amendments and chemical based sanitizers, and cuts growing time.

Using the poultry industry as an example, water and air quality play a significant role in the health and performance of broilers and other poultry production. The poultry industry has concentrated efforts toward improvements on ventilating poultry houses with ambient air, using untreated water supplies, and adding chemicals where necessary to fight disease. The battle between cost and results improvement has always been fought in small incremental improvements, balancing the added cost of treatment versus better growing efficiency. The present invention is an improved system for fighting this battle by delivering better all-around results to growers and integrators, namely, healthier birds, produced in less time, consuming less food and no chemicals.

Overburdening loads of bacteria, viruses and other contaminants (fungi, ammonia and dust levels, etc.) cause birds to grow at below optimum levels. Conversely, reducing the load levels of contaminants in an economical manner benefits the health and production of the birds as well as the profitability of both the grower and the integrator. Ozone is known as a natural bactericide and virucide as well as a chemical free purifier for water and air.

The present invention comprises a combination system for ozonating the air and water provided to a poultry house to provide breathing air and drinking water with less viruses, bacteria and contaminants to the growing poultry. Briefly, a more or less typical air ozonation system provides ozonated air to the poultry house by taking air from outside the poultry house (ambient air), subjecting the ambient air to an ozonation process (ozonated air), and providing the ozonated air to the poultry house. Similarly, a more or less typical water ozonation system provides ozonated water to the poultry house by taking water from outside the poultry house (ambient water), subjecting the ambient water to an ozonation process (ozonated water), and providing the ozonated water to the poultry house. The ozonation process helps eliminate viruses, bacteria and other contaminants in the ambient air and ambient water before the ambient air and ambient water is provided to the poultry house as ozonated air and ozonated water.

The air ozonation system comprises any suitable air ozonation device, including those disclosed in the prior art. The ambient air is ozonated and provided to the poultry house through a series of ducts. Generally, the ozonated air is introduced into the poultry house proximal to the middle of the poultry house and ducted proximal to the ceiling of the poultry house. Vents or registers along the ductwork allow ozonated air to be provided throughout the poultry house.

The water ozonation system comprises any suitable water ozonation device, including those disclosed in the prior art. The ambient water is ozonated and provided to the poultry house through a series of pipes. Generally, the ozonated water is introduced into the poultry house through known poultry drinker systems, such as nipple drinker systems, cup drinking systems, and the like. Drinking stations proximal to the floor of the poultry house allow ozonated drinking water to be provided throughout the poultry house.

The present invention improves the overall performance of the poultry raising process, which is improved significantly for poultry houses treated using the invention. As shown in the accompanying graphs and tables, both average poultry weight and poultry feed conversion results improved consistently in experimental poultry houses utilizing the present invention. In field studies, the poultry houses treated using the present invention were the top performing houses for the grower and integrator for the time period that the birds were harvested.

Generally, one of the invention systems is needed for each grow-out house. Units preferably are installed in a freestanding, weatherproof exterior housing facility at the midline of each house. An ozone delivery is system is installed and the house is ready for increased production.

These features and other features and advantages of the present invention will become more apparent to those of ordinary skill in the relevant art when the following detailed description of the preferred embodiments is read in conjunction with the appended drawings in which like reference numerals designate like components throughout the several views.

### BRIEF DESCRIPTION OF THE DRAWINGS

FIG. 1 is a schematic perspective view of a poultry house with the invention.

FIG. 2 is a schematic view of the air ozonation aspect of the invention.

FIG. 3 is a schematic view of the water ozonation aspect of the invention.

### DETAILED DESCRIPTION OF THE PREFERRED EMBODIMENTS

### 1. System

The present invention comprises a combination system for ozonating the air and water provided to an animal confinement house to provide breathing air and drinking water with less viruses, bacteria and contaminants to the growing animals. Although the invention is applicable to confinement, growing and/or raising houses for all types of animals, for ease of understanding the detailed description of the preferred embodiments will use the poultry industry and poultry houses as a representative example of all animal industries and confinement houses. However, the invention is not meant to be limited only to poultry houses.

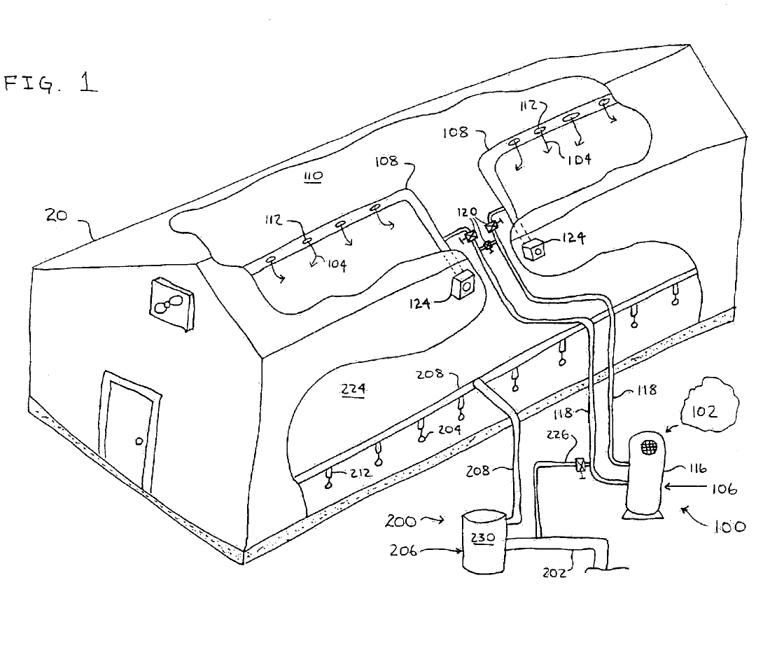

Referring generally to FIG. 1, an air ozonation system 100 provides ozonated air 104 to the poultry house by taking ambient air 102 from outside the poultry house 20, subjecting the ambient air 102 to an ozonation process to produce ozonated air 104, and providing the ozonated air 104 to the poultry house 20. Similarly, a water ozonation system 200 provides ozonated water 204 to the poultry house 20 by taking ambient water 202 from outside the poultry house 20, such as water from a well or the municipal water supply, subjecting the ambient water 202 to an ozonation process to produce ozonated water 204, and providing the ozonated water 204 to the poultry house 20. The ozonation processes 100, 200 help eliminate viruses, bacteria and other contaminants in the ambient air 102 and ambient water 202 before the ambient air 102 and ambient water 202 is provided to the poultry house 20 as ozonated air 104 and ozonated water 204.

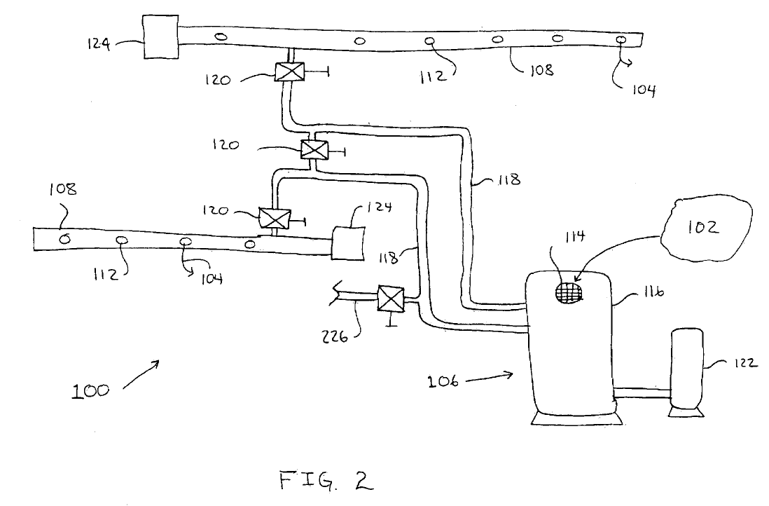

Referring generally to FIG. 1 and specifically to FIG. 2, the air ozonation system 100 is shown in more detail. Generally, the air ozonation system 100 comprises any suitable air ozonation device 106. In general terms, the oxygen in ambient air 102 is ozonated by the ozonation device 106 and ozonated air is provided to the poultry house 20 through ductwork 108. Generally, the ozonated air 104 is introduced into the poultry house 20 proximal to the middle of the poultry house 20 and ducted proximal to the ceiling 110 of the poultry house. Ports, holes, vents or registers 112 along the ductwork 108 allow ozonated air 104 to be provided throughout the poultry house 20.

The air ozonation system 100 comprises an oxygen source, which is preferably oxygen from the ambient air 102 but can be an oxygen supply 122, and an ozone reactor 116 that generates a stream of ozone gas. If ambient air 102 is used as the oxygen source, ozone reactor 116 typically has an input vent 114 through which the ambient air enters the ozone reactor 116. If oxygen supply 122 is used as the oxygen source, oxygen supply 122 can be attached directly to ozone reactor 116. Ozone generated by ozone reactor 116 is provided to ductwork 108 through piping 118. The ozone reactor 116 can be cooled to improve the efficiency of ozone generation.

One or more sets of piping 118 carry ozone to poultry house 20, and one or more sets of ductwork 108 distributes ozonated air 104 within poultry house 20. The illustrative example shown in FIGS. 1 and 2 comprises two sets of piping 118 and two sets of ductwork 108. This allows the level or amount of ozone or ozonated air 104 to be varied in different sections of poultry house 20. Valves 120 can be used to regulate the amount of ozone provided to ductwork 108.

Ductwork 108 directs the ozonated air 104, forced through the ductwork 108 by blowers 124, into the poultry house 20. The details of the air ozonation system 100 are not dealt with or shown in any more detail herein because such is not relevant per se to the present invention and, in addition, suitable ozone generators 116 are known by those ordinarily skilled in the art. The air ozonation system 100 which is referred to herein can be a conventional and commercially available system currently known or developed in the future, or can be a custom-made system. It has been found that ductwork 108 comprising 2-inch diameter tubing (nominally between 1.5 and 2.5 inches in diameter) located within the poultry house 20 is suitable for delivering a sufficient quantity of ozonated air 104 to a conventionally sized poultry house 20.

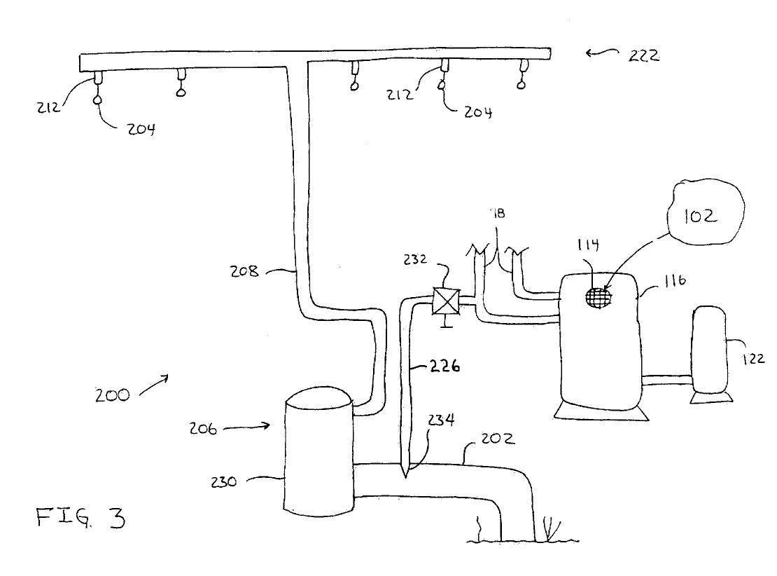

Referring generally to FIG. 1 and specifically to FIG. 3, the water ozonation system 200 is shown in more detail. Generally, the water ozonation system 200 comprises any suitable water ozonation device 206. In general terms, ozone is mixed with ambient water 202 and provided to the poultry house 20 through pipes 208. Generally, the ozonated water 204 is introduced into the poultry house 20 through known poultry drinker systems 222, such as nipple drinker systems, cup drinking systems, and the like. Drinking stations 212 proximal to the floor 224 of the poultry house 20 allow ozonated drinking water 204 to be provided throughout the poultry house 20.

The water ozonation system 200 can and preferably does utilize the same ozone reactor 116 as the air ozonation system 100 to generate a stream of ozone gas. Piping 226 directs ozone from the ozone reactor 116 to the ambient water 202 supply. As shown in FIG. 3, piping 226 can branch off of piping 118, with valve 232 controlling the amount of ozone supplied to the water ozonation system 200. Ozone is injected or mixed with ambient water 202 supply through mixers, injectors or venturis 234, and the ozonated water 204 is directed into a water holding tank 230. The ozonated water 204 is passed through the pipes 208 from the holding tank 230 into the poultry house 20 preferably by the water pressure from the water source or by alternate pressure generating means, such as pumps (not shown). For example, municipal water systems supply ambient water under pressure, and this pressure has been found to be sufficient for the present invention to operate. Likewise, if the ambient water is provided by a well system, such well systems already typically have pumps of sufficient head for the present invention to operate. Alternatively, if necessary, a pump or pumps (not shown) can be provided to provide additional water pressure. The details of the water ozonation system 200 are not dealt with or shown in any more detail herein because such is not relevant per se to the present invention and, in addition, ozone generators are known by those ordinarily skilled in the art. The water ozonation system 200 which is referred to herein is intended to be a conventional and commercially available system currently known or developed in the future, or can be a custom-made system.

Alternatively, each of the air ozonation system 100 and the water ozonation system 200 can have a separate oxygen source and/or ozone reactor 116 designed and optimized for each specific system 100, 200. However, it has been found to be economical and more efficient to use a single oxygen source (ambient air or oxygen supply 122) and ozone generator 116 for the invention.

The present invention improves the overall performance of the poultry raising process. As shown in the following graphs and tables, both average poultry weight and poultry feed conversion results improved consistently in experimental poultry houses utilizing the present invention. In field studies, the poultry houses 20 treated using the present invention had improved performance.

Generally, one combination system comprising one air ozonation system 100 and one water ozonation system 200 is needed for each grow-out poultry house 20. Units preferably are installed in a freestanding, weatherproof exterior housing facility at the midline of each house.

In operation, the ozone reactor 116 generates ozone from the oxygen source. In the air ozonation system, the ozone is directed through piping 118 to ductwork 108, and is then mixed with ambient air 102 and directed through the ductwork 108 within the poultry house 20 for breathing by the birds. The ozone also is mixed with ambient water 202 in the water ozonation system 200 and directed through the pipes 208 into the poultry house 20 drinker system 222 for drinking by the birds. The ozone helps to reduce the quantity of viruses, bacteria and contaminants in the air and water provided to the poultry house 20, resulting in healthier birds, decreased feed conversion ratios, and decreased mortality rates.

### 2. Experimental Results

The following graphs and tables provide experimental results for the present invention. For the following graphs, the white bars represent test results using the invention and the black bars represent control results not using the invention.

a. Feed Conversion.

Using the present invention in a conventional poultry house, the feed conversion of the poultry improved. As the following graph and table show, feed conversion improved for each of the six tests conducted using the present invention when compared to a control sample.

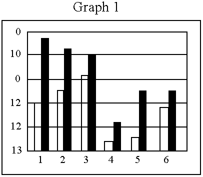

TABLE 1

| Summary Results | | | | | | | | |
|---|---|---|---|---|---|---|---|---|
| AgriO3 | Label | Test 1 | Test 2 | Test 3 | Test 4 | Test 5 | Table 6 | Average |
| Feed Conversion | Test | 1.75 | 1.78 | 1.81 | 1.67 | 1.68 | 1.74 | 1.74 |
| Feed Converstion | Control | 1.89 | 1.87 | 1.85 | 1.71 | 1.78 | 1.78 | 1.81 |

b. Average Weight.

Using the present invention in a conventional poultry house, the average weight of the poultry improved. As the following graph and table show, average weight improved for five of the six tests conducted using the present invention when compared to a control sample.

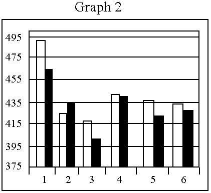

TABLE 2

| Summary Results | | | | | | | | |
|---|---|---|---|---|---|---|---|---|
| AgriO3 | Label | Test 1 | Test 2 | Test 3 | Test 4 | Test 5 | Test 6 | Average |
| Average Weight | Test | 4.91 | 4.29 | 4.17 | 4.46 | 4.37 | 4.33 | 4.42 |
| Average Weight | Control | 4.67 | 4.36 | 4.04 | 4.42 | 4.22 | 4.29 | 4.33 |

The numbers shown in Table 2 are pounds.

c. Average Weekly Cost.

Using the present invention in a conventional poultry house, the average weekly cost of operating a conventional poultry house improved. As the following graph and table show, average weekly cost of operating improved for each of the six tests conducted using the present invention when compared to a control sample. For example, in Test 1, using the present invention, the grower would receive an additional 0.78 cents per pound above contract terms at the end of grow out, while under control conditions (that is, without using the present invention), the grower would receive 0.65 cents per pound below contract terms at the end of grow out.

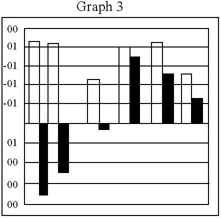

TABLE 3

| Summary Results | | | | | | | | |
|---|---|---|---|---|---|---|---|---|
| AgriO3 | Label | Test 1 | Test 2 | Test 3 | Test 4 | Test 5 | Test 6 | Average |
| Earnings Bonus | Test | −0.78 | −0.76 | −0.42 | −0.7 | −0.73 | −0.43 | −0.64 |
| Earnings Bonus | Control | 0.65 | 0.53 | 0.07 | −0.57 | −0.44 | −0.14 | −0.01 |

The numbers shown in Table 3 are fractions of cents.

As shown in the general overall schematic diagram of FIG. 1, operation of the present invention involves the generation of ozone, the mixture of a portion of the ozone with ambient air 102 and a portion of the ozone with ambient water 202, the transport and distribution of the ozonated air 104 and the ozonated water 204 to an animal confinement house 20, and the release of the ozonated air 104 and the ozonated water 204 into the animal confinement house 20 via overhead ductwork 108 for the ozonated air 104 and a drinking system 222 for the ozonated water 204. The ozone reactor 116 receives an input stream of oxygen from the oxygen source and produces, through a chemical reaction process not detailed herein, at least one output stream of ozone gas.

The above detailed description of the preferred embodiments, examples, and the appended figures are for illustrative purposes only and are not intended to limit the scope and spirit of the invention, and its equivalents, as defined by the appended claims. For example purposes only, the ductwork 108 can be located at any suitable position throughout the animal house 20, and not necessarily overhead; the ozone reactor 116 can be any source of ozone; additional additives, such as vitamins and antibiotics, can be added to the ozonated air 104 and/or ozonated water 204, for the health of the animals; and the like. One skilled in the art will recognize that many variations can be made to the invention disclosed in this specification without departing from the scope and spirit of the invention.

What is claimed is:

1. A system for ozonating air and water provided to an animal confinement house, comprising:
a. an ozone generator for generating ozone;
b. a means for mixing at least a portion of the ozone with ambient air to produce ozonated air;
c. a means for mixing at least another portion of the ozone with ambient water to produce ozonated water;
d. a means for directing the ozonated air into the animal confinement house for consumption by animals within the animal confinement house; and
e. a means for directing the ozonated water into the animal confinement house for consumption by the animals within the animal confinement house.

2. The system for ozonating air and water as claimed in claim 1, wherein the ambient air is supplied from outside the animal confinement house, and the at least a portion of the ozone is mixed with the ambient air from outside the animal confinement house to produce the ozonated air.

3. The system for ozonating air and water as claimed in claim 2, wherein the means for directing the ozonated air into the animal confinement house further comprises ductwork fluidly connecting the ozone generator to the animal confinement house and means for introducing the ozonated air through the ductwork and into the animal confinement house.

4. The system for ozonating air and water as claimed in claim 3, wherein the ductwork comprises means for allowing the ozonated air to be introduced into the animal confinement house.

5. The system for ozonating air and water as claimed in claim 1, wherein the ambient water is supplied from outside the animal confinement house, and the at least another portion of the ozone is mixed with the ambient water from outside the animal confinement house in a means for ozonating water to produce the ozonated water.

6. The system for ozonating air and water as claimed in claim 5, wherein the means for directing the ozonated water into the animal confinement house further comprises pipes fluidly connecting the means for ozonating water to the animal confinement house and means for introducing the ozonated water through the pipes and into the animal confinement house.

7. The system for ozonating air and water as claimed in claim 6, wherein the pipes terminate in a drinking system for allowing the ozonated water to be provided to animals in the animal confinement house.

8. The system for ozonating air and water as claimed in claim 1, wherein the ozone generator further comprises an oxygen supply from which the ozone is generated.

9. The system for ozonating air and water as claimed in claim 8, wherein the oxygen supply is ambient air.

10. The system for ozonating air and water as claimed in claim 1, further comprising a means for cooling the ozone reactor.

11. A system for ozonating air and water provided to an animal confinement house, comprising:
a. an ozone generator for generating ozone;
b. a means for mixing at least a portion of the ozone with ambient air supplied from outside the animal to produce ozonated air;
c. a means for mixing at least another portion of the ozone with ambient water supplied from outside the animal confinement house to produce ozonated water;
d. a means for directing the ozonated air into the animal confinement house for consumption by animals within the animal confinement house, wherein the means for directing the ozonated air into the animal confinement house further comprises ductwork fluidly connecting the ozone generator to the animal confinement house and means for introducing the ozonated air through the ductwork and into the animal confinement house; and
e. a means for directing the ozonated water into the animal confinement house for consumption by the animals within the animal confinement house, wherein the means for directing the ozonated water into the animal confinement house further comprises pipes fluidly connecting the means for mixing at least another portion of the ozone with ambient water to the animal confinement house and means for introducing the ozonated water through the pipes and into the animal confinement house.

12. The system for ozonating air and water as claimed in claim 11, wherein the ductwork comprises ports for allowing the ozonated air to be introduced into the animal confinement house, and wherein the pipes terminate in a drinking system for allowing the ozonated water to be provided to animals in the animal confinement house.

13. The system for ozonating air and water as claimed in claim 12, further comprising an oxygen supply from which the ozone is generated, the oxygen supply being ambient air.

14. The system for ozonating air and water as claimed in claim 13, wherein the animal confinement house is a poultry house.

15. A process for ozonating the air and water provided to an animal confinement house, comprising:
a. producing ozone;
b. mixing at least a portion of the ozone with ambient air to produce ozonated air;
c. mixing at least another portion of the ozone with ambient water to produce ozonated water;
d. directing the ozonated air into the animal confinement house for consumption by animals within the animal confinement house; and
e. directing the ozonated water into the animal confinement house for consumption by the animals within the animal confinement house.

16. The process for ozonating air and water as claimed in claim 15, wherein the ozone is fed to an ozone generator, and the at least a portion of the ozone is mixed with ambient air from outside the animal confinement house to produce the ozonated air.

17. The process for ozonating air and water as claimed in claim 16, wherein the ozonated air is introduced into the animal confinement house through ductwork fluidly connecting the ozone generator to the animal confinement house.

18. The process for ozonating air and water as claimed in claim 17, wherein the ductwork comprises vents allowing the ozonated air to be introduced into the animal confinement house.

19. The process for ozonating air and water as claimed in claim 11, wherein the ozone is fed to an ozone generator, and the at least another portion of the ozone is mixed in a means for ozonating water with ambient water from outside the animal confinement house to produce the ozonated water.

20. The process for ozonating air and water as claimed in claim 19, wherein the ozonated water is introduced into the animal confinement house through pipes fluidly connecting the means for ozonating water to the animal confinement house.

21. The process for ozonating air and water as claimed in claim 20, wherein the pipes terminate in a drinking system allowing the ozonated water to be provided to animals in the animal confinement house.

22. The process for ozonating air and water as claimed in claim 21, wherein the ozone reactor generates ozone from an oxygen supply.

23. The process for ozonating air and water as claimed in claim 22, wherein the oxygen supply ambient air.

24. The process for ozonating air and water as claimed in claim 15, further comprising cooling the ozone generator when generating the ozone.

25. A process for ozonating air and water provided to an animal confinement house, comprising:
a. generating ozone;
b. mixing at least a portion of the ozone with ambient air supplied from outside the animal confinement house to produce ozonated air;
c. mixing in a means for ozonating water at least another portion of the ozone with ambient water supplied from outside the animal confinement house to produce ozonated water;
d. directing the ozonated air into the animal confinement house for consumption by animals within the animal confinement house by directing the ozonated air into the animal confinement house through ductwork fluidly connecting the ozone generator to the animal confinement house; and
e. directing the ozonated water into the animal confinement house for consumption by the animals within the animal confinement house by directing the ozonated water into the animal confinement house through pipes fluidly connecting the means of ozonating water to the animal confinement house.

26. The process for ozonating air and water as claimed in claim 25, wherein the ductwork comprises vents allowing the ozonated air to be introduced into the animal confinement house, and wherein the pipes terminate in a drinking system allowing the ozonated water to be provided to animals in the animal confinement house.

27. The process for ozonating air and water as claimed in claim 26, wherein the ozone is generated from oxygen in ambient air.

28. The process for ozonating air and water as claimed in claim 25, wherein the animal confinement house is a poultry house.
